(19) United States
(12) Patent Application Publication
(10) Pub. No.: US 2021/0401765 A1
(43) Pub. Date: Dec. 30, 2021

(54) SPRAY COMPOSITIONS OF CHITOSAN FOR WOUND HEALING

(71) Applicant: SHILPA MEDICARE LIMITED, RAICHUR (IN)

(72) Inventors: PURUSHOTHAMA NARASIYAPPA, Bengaluru (IN); KIRAN KUMAR CHERUKURI, Bengaluru (IN); VIVEK VIJAYA KUMAR JOSHI, Bengaluru (IN); PRADEEP SHIVAKUMAR, Bengaluru (IN); SREENIVASA REDDY, Bengaluru (IN)

(73) Assignee: SHILPA MEDICARE LTD, RAICHUR (IN)

(21) Appl. No.: 16/618,121

(22) PCT Filed: Oct. 18, 2019
(86) PCT No.: PCT/IB2019/058890

§ 371 Date: Nov. 28, 2019

(30) Foreign Application Priority Data
Oct. 27, 2018 (IN) 201841040573

(51) Int. Cl. A61K 9/70; A61K 9/12; A61K 31/722; A61K 31/265; A61K 31/191

(52) U.S. Cl. CPC A61K 9/7015; A61K 9/12; A61K 31/191; A61K 31/265; A61K 31/722

(57) ABSTRACT

The present invention relates to topical spray compositions comprising chitosan and the process for preparation thereof. The topical spray compositions comprising chitosan are used for the treating burn wounds, wounds resulting from chemical burns, wounds from physical trauma, neuropathic ulcers, pressure sores, diabetic ulcers. Further the spray compositions composition is used for treating gynaecological diseases, urogenital infections (vaginal bacteriosis, urinary tract infection and vaginitis) with good effect, safe, natural, and good biocompatibility by restoring and/or maintaining the pH of vagina. More specifically the present invention relates to topical spray compositions comprising chitosan and carbonic acid and the process for preparation thereof.

### FIELD OF INVENTION

The present invention relates to topical spray compositions for treatment of wound healing comprising chitosan and the process for preparation thereof. Further the present invention relates to topical spray compositions for treating gynaecological diseases, urogenital infections (vaginal bacteriosis, urinary tract infection and vaginitis) with good effect, safe, natural, and good biocompatibility by restoring and/or maintaining the pH of vagina. More specifically the present invention discloses topical spray composition comprising chitosan and carbonic acid. Even more specifically the present invention discloses the spray composition comprising chitosan, lactic acid, carbonic acid and water stored in a high-pressure resistant container bottle with a nozzle in a canning mode or high-pressure resistant Bag-On-Valve (BOV) aerosol dispensing system. Most specifically the present invention discloses the topical spray composition comprising chitosan, lactic acid, carbonic acid and water stored in high-pressure resistant Bag-On-Valve (BOV) aerosol dispensing system.

### BACKGROUND OF INVENTION

The primary goal in the treatment of wounds is to achieve wound closure. Open cutaneous wounds represent one major category of wounds and include burn wounds, wounds resulting from chemical burns, wounds from physical trauma, neuropathic ulcers, pressure sores and diabetic ulcers. The most commonly used conventional modality to assist in wound healing involves the usage of wound dressings primarily gauze, cotton wool, bandages and the like.

Chitosan is the natural polysaccharide have a good biocompatibility, biodegradability and promote wound healing. Chitosan wound treatment, can promote cell proliferation, blood clotting and tissue growth. Therefore, chitosan wound dressings have gained good attention for treatment of wounds.

U.S. Pat. No. 7,897,832 discloses a wound dressing comprising chitosan biomaterial that has been freeze-dried and heat compressed to form a hydrophilic sponge structure for the treatment of wounds. This chitosan dressing is in a sponge form which uses a large area on the wound surface on body part, poor adhesion with the wound, and further inconvenient to store, which needs the large area for storage.

In order to overcome the above disadvantages spray composition containing chitosan are developed as disclosed in CN104856978B and CN106512081A.

CN 104856978B discloses the chitosan spray agent comprising of about 0.05% to about chitosan, from 0.01% to 0.15% lactic acid, 0.1% to 0.2% surfactant, 0.1% to 0.3% cosolvent and water, which is directly sprayed on to wound surface to form a film.

CN106512081A discloses a spray type wound dressing comprising chitosan, glycerin, carbon dioxide and water, which is stored into a high-pressure resistant container bottle with a nozzle in a canning mode. This patent application discloses the chitosan in range of 1% to about 5% and carbon dioxide in the range of 20% to about 30%, wherein chitosan forms the gel as sprayed onto the wound surface. It is very difficult to manufacture and handle the canister which contains about 1% to about 5% of chitosan and about 20% to about 30% of carbon dioxide.

Urogenital infections (vaginal bacteriosis, urinary tract infection and vaginitis) affect more than one billion women per year, all over the world, representing the main reason for women to seek medical consultation. The three main causes of vaginal problems are trichomoniasis, vulvovaginal candidosis and vaginal bacteriosis. Vaginal bacteriosis is a complex condition, occurring due to change in the normal vaginal flora dominated by Lactobacillus spp., for a polymicrobial community of other aerobes and anaerobes, namely Prevotella spp., Mobilluncus spp., Gardenerella vaginalis and Mycoplasma hominis. The decrease in the number of Lactobacillus spp., microorganisms responsible for the production of lactic acid that confers acidic pH (3.5-4.5) to the vaginal milieu, explains in this affection, the usually high pH. To control the bacterial growth, the vagina is normally slightly acidic with a pH of 3.8 to 4.2 and the pH greater than 4.5 is considered alkaline and is suggestive of bacterial vaginosis. A vaginal bacteriosis situation is characterized by excessive vaginal discharge produced, pH increase and unpleasant odor, being therefore associated with high discomfort. This infection is related to disturbances of the normal flora has raised the interest in using protective microorganisms (probiotics) to recolonize the vagina in the treatment and prevention of recurrence.

US Patent Application No. 20170224749A1 discloses the composition of aqueous base for a film/solid foam for use in the treatment of urogenital infections comprising plasticizer, at least a mucoadhesive polymer, at least a probiotic and/or prebiotic, and at least an active substance in pharmacologically effective amounts.

CN102688182B discloses the antibacterial vaginal gel containing polycarbophil, carbomer, EDTA, chitosan, glycerol, triethanolamine, methyl paraben and deionized water with a pH value of 3.8 to 4.4.

CN103877452B discloses a vaginal rinse liquid comprising chitosan, glycerol, alum in an aqueous system.

In order to overcome disadvantages for treatment of wounds and urogenital infections, there exists a need to further develop a topical spray composition comprising chitosan and carbonic acid stored in a high-pressure resistant container bottle with a nozzle in a canning mode or a high-pressure resistant Bag-On-Valve (BOV) aerosol dispensing system, more specifically the spray composition is stored in high-pressure resistant Bag-On-Valve (BOV) aerosol dispensing system. The inventors of present invention have developed topical spray compositions containing about 0.01% w/v to about 1.0% w/v of chitosan and about 0.1% w/v to about 1.0% w/v of carbonic acid in high-pressure resistant Bag-On-Valve (BOV) aerosol dispensing system, and present inventors have surprisingly found that wound healing rate of present invention was significantly more when compared to chitosan or carbonated solution sprayed on wound individually. Further the inventors of the present invention have surprisingly found the synergistic activity for the treatment of vaginal bacteriosis with the topical spray composition comprising chitosan and carbonic acid, wherein the pH of the composition is of about 2.0 to about 4.5 stored in a Bag-On-Valve (BOV) aerosol dispensing system.

### SUMMARY OF THE INVENTION

In first aspect the present invention relates to the topical spray composition comprising chitosan and carbonic acid.

In one embodiment the topical spray composition of the present invention comprising chitosan and carbonic acid is in the form of a solid foam or film when applied on to the surface of the skin.

In another embodiment the topical spray composition of the present invention comprising chitosan and carbonic acid is used for the treating burn wounds, wounds resulting from chemical burns, wounds from physical trauma, neuropathic ulcers, pressure sores and diabetic ulcers.

In a further embodiment the topical spray composition of the present invention comprising chitosan and carbonic acid is used for the treatment of Urogenital infections (vaginal bacteriosis, urinary tract infection and vaginitis) and maintenance of vaginal pH.

In embodiments of the invention, the present invention relates to the topical spray composition comprising about 0.01% w/v to about 1.0% w/v of chitosan and about 0.1% w/v to about 1.0% w/v of carbonic acid.

In another embodiment of the invention, the present invention relates to the topical spray composition comprising about 0.01% w/v to about 1.0% w/v of chitosan and about 0.1% w/v to about 1.0% w/v of carbonic acid, wherein spray composition is stored in a pressurized vessel.

In a further embodiment of the invention, the present invention relates to the topical spray composition comprising about 0.01% w/v to about 1.0% w/v of chitosan and about 0.1% w/v to about 1.0% w/v of carbonic acid, wherein spray composition is stored in high-pressure resistant container bottle with a nozzle in a canning mode.

In a still further embodiment of the invention, the present invention relates to the topical spray composition comprising about 0.01% w/v to about 1.0% w/v of chitosan and about 0.1% w/v to about 1.0% w/v of carbonic acid, wherein spray composition is stored in high-pressure resistant Bag-On-Valve (BOV) aerosol dispensing system.

In a specific embodiment of the invention, the present invention relates to the topical spray composition comprising about 0.05% w/v to about 0.1% w/v of chitosan and about 0.2% w/v to about 0.4% w/v of carbonic acid.

In one more embodiment of the invention, the present invention relates to the topical spray composition comprising about 0.05% w/v to about 0.1% w/v of chitosan and about 0.2% w/v to about 0.4% w/v of carbonic acid, wherein the spray composition is stored in a pressurized vessel.

In another embodiment of the invention, the present invention relates to the topical spray composition comprising about 0.05% w/v to about 0.1% w/v of chitosan and about 0.2% w/v to about 0.4% w/v of carbonic acid, wherein the spray composition is stored in high-pressure resistant container bottle with a nozzle in a canning mode.

In a further embodiment of the invention, the present invention relates to the topical spray composition comprising about 0.05% w/v to about 0.1% w/v of chitosan and about 0.2% w/v to about 0.4% w/v of carbonic acid, wherein the spray composition is stored in high-pressure resistant Bag-On-Valve (BOV) aerosol dispensing system.

In second aspect the present invention further relates to the topical spray composition comprising chitosan, carbonic acid and water.

In embodiments of the invention, the present invention relates to the topical spray composition comprising chitosan, carbonic acid and water, wherein spray composition is stored in a pressurized vessel.

In another embodiment of the invention, the present invention relates to the topical spray composition comprising chitosan, carbonic acid and water, wherein spray composition is stored in high-pressure resistant container bottle with a nozzle in a canning mode.

In further embodiment of the invention, the present invention relates to the topical spray composition comprising chitosan, carbonic acid and water, wherein spray composition is stored in high-pressure resistant Bag-On-Valve (BOV) aerosol dispensing system.

In a further embodiment of the invention, the present invention relates to the topical spray composition comprising about 0.01% w/v to about 1.0% w/v of chitosan, about 0.1% w/v to about 1.0% w/v of carbonic acid and water.

In another embodiment of the invention, the present invention relates to the topical spray composition comprising about 0.01% w/v to about 1.0% w/v of chitosan, about 0.1% w/v to about 1.0% w/v of carbonic acid and water, wherein spray composition is stored in a pressurized vessel.

In a further embodiment of the invention, the present invention relates to the topical spray composition comprising about 0.01% w/v to about 1.0% w/v of chitosan, about 0.1% w/v to about 1.0% w/v of carbonic acid and water, wherein spray composition is stored in high-pressure resistant container bottle with a nozzle in a canning mode.

In a still further embodiment of the invention, the present invention relates to the topical spray composition comprising about 0.01% w/v to about 1.0% w/v of chitosan, about 0.1% w/v to about 1.0% w/v of carbonic acid and water, wherein spray composition is stored in high-pressure resistant Bag-On-Valve (BOV) aerosol dispensing system.

In third aspect the present invention relates to the topical spray composition comprising chitosan, lactic acid, carbonic acid and water.

In one embodiment, the present invention relates to the topical spray composition comprising chitosan, lactic acid, carbonic acid and water, wherein spray composition is stored in a pressurized vessel.

In another embodiment, the present invention relates to the topical spray composition comprising chitosan, lactic acid, carbonic acid and water, wherein spray composition is stored in high-pressure resistant container bottle with a nozzle in a canning mode.

In a further embodiment, the present invention relates to the topical spray composition comprising chitosan, lactic acid, carbonic acid and water, wherein spray composition is stored in high-pressure resistant Bag-On-Valve (BOV) aerosol dispensing system.

In another embodiment of the invention, the present invention relates to the topical spray composition comprising about 0.01% w/v to about 1.0% w/v of chitosan, about 0.01% w/v to 2.0% w/v of lactic acid, about 0.1% w/v to about 1.0% w/v of carbonic acid and water.

In a further embodiment of the invention, the present invention relates to the topical spray composition comprising about 0.01% w/v to about 1.0% w/v of chitosan, about 0.01% w/v to 2.0% w/v of lactic acid, about 0.1% w/v to about 1.0% w/v of carbonic acid and water, wherein the spray composition is stored in a pressurized vessel.

In another embodiment of the invention, the present invention relates to the topical spray composition comprising about 0.01% w/v to about 1.0% w/v of chitosan, about 0.01% w/v to 2.0% w/v of lactic acid, about 0.1% w/v to about 1.0% w/v of carbonic acid and water, wherein spray composition is stored in high-pressure resistant container bottle with a nozzle in a canning mode.

In a still further embodiment of the invention, the present invention relates to the topical spray composition comprising about 0.01% w/v to about 1.0% w/v of chitosan, about 0.01% w/v to 2.0% w/v of lactic acid, about 0.1% w/v to about 1.0% w/v of carbonic acid and water, wherein spray composition is stored in in high-pressure resistant Bag-On-Valve (BOV) aerosol dispensing system.

In a further embodiment of the invention, the present invention relates to the topical spray composition comprising about 0.01% w/v to about 1.0% w/v of chitosan, about 0.01% w/v to 2.0% w/v of lactic acid, about 0.1% w/v to about 1.0% w/v of carbonic acid and water quantity sufficient to 100% w/v of composition.

In another embodiment of the invention, the present invention relates to the topical spray composition comprising about 0.01% w/v to about 1.0% w/v of chitosan, about 0.01% w/v to 0.2% w/v of lactic acid, about 0.1% w/v to about 1.0% w/v of carbonic acid and water, wherein the spray composition is stored in a pressurized vessel.

In a further embodiment of the invention, the present invention relates to the topical spray composition comprising about 0.01% w/v to about 1.0% w/v of chitosan, about 0.01% w/v to 0.2% w/v of lactic acid, about 0.1% w/v to about 1.0% w/v of carbonic acid and water, wherein the spray composition is stored in high-pressure resistant container bottle with a nozzle in a canning mode.

In a still further embodiment of the invention, the present invention relates to the topical spray composition comprising about 0.01% w/v to about 1.0% w/v of chitosan, about 0.01% w/v to 0.2% w/v of lactic acid, about 0.1% w/v to about 1% w/v of carbonic acid and water, wherein spray composition is stored in in high-pressure resistant Bag-On-Valve (BOV) aerosol dispensing system.

In another of the invention, the present invention relates to the topical spray composition comprising about 0.01% w/v to about 1.0% w/v of chitosan, about 0.01% w/v to 2.0% w/v of lactic acid, about 0.1% w/v to about 1.0% w/v of carbonic acid and water quantity sufficient to 100% w/v of composition, wherein the spray composition has a pH of about 2.0 to about 4.5.

In another embodiment of the invention, the present invention relates to the topical spray composition comprising about 0.01% w/v to about 1.0% w/v of chitosan, about 0.01% w/v to 0.2% w/v of lactic acid, about 0.1% w/v to about 1.0% w/v of carbonic acid and water, wherein the spray composition has a pH of about 2.0 to about 4.5 and wherein the spray composition is stored in a pressurized vessel.

In a further embodiment of the invention, the present invention relates to the topical spray composition comprising about 0.01% w/v to about 1.0% w/v of chitosan, about 0.01% w/v to 0.2% w/v of lactic acid, about 0.1% w/v to about 1.0% w/v of carbonic acid and water, wherein the spray composition has a pH of about 2.0 to about 4.5 and wherein the spray composition is stored in high-pressure resistant container bottle with a nozzle in a canning mode.

In a still further embodiment of the invention, the present invention relates to the topical spray composition comprising about 0.01% w/v to about 1.0% w/v of chitosan, about 0.01% w/v to 0.2% w/v of lactic acid, about 0.1% w/v to about 1% w/v of carbonic acid and water, wherein the spray composition has a pH of about 2.0 to about 4.5 and wherein spray composition is stored in in high-pressure resistant Bag-On-Valve (BOV) aerosol dispensing system.

### BRIEF DESCRIPTION OF THE DRAWINGS

FIG. 1 discloses the rate of wound contraction (healing) calculated of the present inventive composition [chitosan in carbonic acid and water (carbonated water) as disclosed in Example-1] in comparison to chitosan solution (without carbonation), carbonic acid in water (carbonated water), reference and normal control (untreated).

FIG. 2 discloses the average wound breaking strength of Example-1 in comparison to chitosan solution (without carbonation), carbonated water, reference and normal control (untreated).

### DETAILED DESCRIPTION OF THE INVENTION

The present invention relates to the topical spray composition comprising chitosan and carbonic acid.

In a specific embodiment, the present invention contains chitosan. The molecular weight of the chitosan used in the present invention is in range of about 50,000 to about 100,000. In the topical spray composition, chitosan is present in the range of about 0.01% w/v to about 1.0% w/v. More preferably chitosan used in the topical spray composition is about 0.01% w/v, 0.015% w/v, 0.02% w/v, 0.025% w/v, 0.03% w/v, 0.04% w/v, 0.045% w/v, 0.05% w/v, 0.055% w/v, 0.06% w/v, 0.065% w/v, 0.07% w/v, 0.075% w/v, 0.08% w/v, 0.085% w/v, 0.09% w/v, 0.095% w/v, 0.1% w/v, 0.15% w/v, 0.20% w/v, 0.25% w/v, 0.30% w/v, 0.40% w/v, 0.50% w/v, 0.60% w/v, 0.70% w/v, 0.75% w/v, 0.80% w/v, 0.90% w/v and 1.0% w/v. Most preferably chitosan used in the topical spray composition is of about 0.05% w/v to about 0.1% w/v and even most preferably chitosan used in the topical spray composition is of about 0.05% w/v, 0.055% w/v, 0.06% w/v, 0.065% w/v, 0.07% w/v, 0.075% w/v, 0.08% w/v, 0.085% w/v, 0.09% w/v, 0.095% w/v, 0.1% w/v.

In embodiments of the present invention, the inventive topical spray composition comprises carbonic acid. Carbonic acid used in the present invention is in range of about 0.1% w/v to about 1.0% w/v. More preferably carbonic acid used in the spray composition is about 0.2% w/v to about 0.4% w/v of carbonic acid and most preferably carbonic acid used in the spray composition is of about 0.2% w/v, 0.25% w/v, 0.3% w/v, 0.35% w/v and 0.4% w/v.

The present invention further provides the topical spray composition comprising chitosan, carbonic acid, lactic acid and water.

In embodiments of the present invention, the inventive spray composition comprises lactic acid. Lactic acid is used to dissolve the chitosan. Lactic acid used in the present invention is in range of about 0.01% w/v to about 2.0% w/v. More preferably lactic acid used in the topical spray composition is about 0.01% w/v, 0.02% w/v, 0.03% w/v, 0.04% w/v, 0.05% w/v, 0.055% w/v, 0.06% w/v, 0.065% w/v, 0.07% w/v, 0.075% w/v, 0.08% w/v, 0.085% w/v, 0.09% w/v, 0.095% w/v, 0.1% w/v, 0.15% w/v, 0.20% w/v, 0.25% w/v, 0.30% w/v, 0.40% w/v, 0.50% w/v, 0.60% w/v, 0.70% w/v, 0.75% w/v, 0.80% w/v, 0.90% w/v and 1.0% w/v, 1.05% w/v, 1.1% w/v, 1.15% w/v, 1.2% w/v, 1.25% w/v, 1.3% w/v, 1.35% w/v, 1.4% w/v, 1.45% w/v, 1.5% w/v, 1.55% w/v, 1.6% w/v, 1.65% w/v, 1.7% w/v, 1.75% w/v, 1.8% w/v, 1.85% w/v, 1.9% w/v, 1.95% w/v and 2.0% w/v.

In embodiments of the present invention, the inventive spray composition comprises water. Water used in the present composition quantity sufficient to 100% w/v of composition.

In embodiments of the present invention, the present invention, the topical spray composition comprises chitosan, carbonic acid, lactic acid and water, wherein the topical spray composition has a pH of about 2.0 to about 4.5.

In further embodiments of the invention, the present invention provides the topical spray composition comprising additional excipients selected from glycerin. Glycerin is preferably used in the range from about 0.1% w/v to 1% w/v of the composition and more preferably 0.2% w/v, 0.3% w/v, 0.4% w/v, 0.5% w/v, 0.6% w/v, 0.7% w/v and 0.8% w/v.

In embodiments of the present invention, the present invention provides the topical spray composition comprising chitosan and carbonic acid stored in a pressurized vessel.

In further embodiments of the present invention, the present invention provides the topical spray composition comprising chitosan, carbonic acid, lactic acid and water stored in a pressurized vessel.

In embodiments of the invention, carbonic acid gas continuously introduced into water, carbonic acid gas is dissolved in water to form carbonic acid solution. The topical spray composition according to the present invention is stored in a pressure vessel. Higher pressures may facilitate the dissolved carbonic acid in solution to form a stable solution of chitosan. The topical spray composition of the present invention was charged in vials of high pressure for portable use. By shaking the pressurized container, the solution of carbonic acid gas released, after the discharge nozzle, spray composition quickly escape, which forms a solid film or film on the wound surface for the treating burn wounds, wounds resulting from chemical burns, wounds from physical trauma, neuropathic ulcers, pressure sores and diabetic ulcers or surface of skin for the treatment of urogenital infections (vaginal bacteriosis, urinary tract infection and vaginitis). In embodiments of the invention the pressurized vessel used is high-pressure resistant container bottle with a nozzle in a canning mode (where the composition of the present invention is mixed with propellant like nitrogen and filled into container and sealed) or high-pressure resistant Bag-On-Valve (BOV) aerosol dispensing system. Most preferably the pressurized vessel used in the present invention is Bag-On-Valve (BOV) aerosol dispensing system.

In the embodiments of the present invention, the composition comprising chitosan, carbonic acid, lactic acid and water is mixed with propellant like nitrogen and/or compressed air and filled into the high-pressure resistant container bottle with a nozzle in canning mode.

In another embodiment of the present invention, the composition comprising chitosan, carbonic acid, lactic acid and water if filled into the bag, the bag is fitted into the pressurized container containing propellant like nitrogen and/or compressed air and sealed to fitted with actuator to form the bag-on-valve (BOV) aerosol dispensing system.

In embodiments of the invention, the present invention provides the topical spray composition consisting essentially about 0.01% w/v to about 1.0% w/v of chitosan and about 0.1% w/v to about 1.0% w/v of carbonic acid.

In another embodiment of the invention, the present invention provides the topical spray composition consisting essentially about 0.01% w/v to about 1.0% w/v of chitosan and about 0.1% w/v to about 1.0% w/v of carbonic acid, wherein spray composition is stored in a pressurized vessel.

In a further embodiment of the invention, the present invention provides the topical spray composition consisting essentially about 0.01% w/v to about 1.0% w/v of chitosan and about 0.1% w/v to about 1.0% w/v of carbonic acid, wherein spray composition is stored in high-pressure resistant container bottle with a nozzle in a canning mode.

In a still further embodiment of the invention, the present invention provides the topical spray composition consisting essentially about 0.01% w/v to about 1.0% w/v of chitosan and about 0.1% w/v to about 1.0% w/v of carbonic acid, wherein spray composition is stored in high-pressure resistant Bag-On-Valve (BOV) aerosol dispensing system.

In a specific embodiment of the invention, the present invention provides the topical spray composition consisting essentially about 0.05% w/v to about 0.1% w/v of chitosan and about 0.2% w/v to about 0.4% w/v of carbonic acid.

In one more embodiment of the invention, the present invention provides the topical spray composition consisting essentially about 0.05% w/v to about 0.1% w/v of chitosan and about 0.2% w/v to about 0.4% w/v of carbonic acid, wherein the spray composition is stored in a pressurized vessel.

In another embodiment of the invention, the present invention provides the topical spray composition consisting essentially about 0.05% w/v to about 0.1% w/v of chitosan and about 0.2% w/v to about 0.4% w/v of carbonic acid, wherein the spray composition is stored in high-pressure resistant container bottle with a nozzle in a canning mode.

In a further embodiment of the invention, the present invention provides the topical spray composition consisting essentially about 0.05% w/v to about 0.1% w/v of chitosan and about 0.2% w/v to about 0.4% w/v of carbonic acid, wherein the spray composition is stored in high-pressure resistant Bag-On-Valve (BOV) aerosol dispensing system.

In embodiments of the invention, the present invention provides the topical spray composition consisting about 0.01% w/v to about 1.0% w/v of chitosan, about 0.01% w/v to 2.0% w/v of lactic acid, about 0.1% w/v to about 1.0% w/v of carbonic acid and water.

In a further embodiment of the invention, the present invention provides the topical spray composition consisting about 0.01% w/v to about 1.0% w/v of chitosan, about 0.01% w/v to 2.0% w/v of lactic acid, about 0.1% w/v to about 1.0% w/v of carbonic acid and water, wherein the spray composition is stored in a pressurized vessel.

In another embodiment of the invention, the present invention provides the topical spray composition consisting about 0.01% w/v to about 1.0% w/v of chitosan, about 0.01% w/v to 2.0% w/v of lactic acid, about 0.1% w/v to about 1.0% w/v of carbonic acid and water, wherein spray composition is stored in high-pressure resistant container bottle with a nozzle in a canning mode.

In a still further embodiment of the invention, the present invention provides the topical spray composition consisting about 0.01% w/v to about 1.0% w/v of chitosan, about 0.01% w/v to 2.0% w/v of lactic acid, about 0.1% w/v to about 1.0% w/v of carbonic acid and water, wherein spray composition is stored in in high-pressure resistant Bag-On-Valve (BOV) aerosol dispensing system.

In a further embodiment of the invention, the present invention provides the topical spray composition consisting about 0.01% w/v to about 1.0% w/v of chitosan, about 0.01% w/v to 2.0% w/v of lactic acid, about 0.1% w/v to about 1.0% w/v of carbonic acid and water quantity sufficient to 100% w/v of composition.

In another embodiment of the invention, the present invention provides the topical spray composition consisting about 0.01% w/v to about 1.0% w/v of chitosan, about 0.01% w/v to 0.2% w/v of lactic acid, about 0.1% w/v to about 1.0% w/v of carbonic acid and water, wherein the spray composition is stored in a pressurized vessel.

In a further embodiment of the invention, the present invention provides the topical spray composition consisting about 0.01% w/v to about 1.0% w/v of chitosan, about 0.01% w/v to 0.2% w/v of lactic acid, about 0.1% w/v to about 1.0% w/v of carbonic acid and water, wherein the spray composition is stored in high-pressure resistant container bottle with a nozzle in a canning mode.

In a still further embodiment of the invention, the present invention provides to the topical spray composition consisting about 0.01% w/v to about 1.0% w/v of chitosan, about 0.01% w/v to 0.2% w/v of lactic acid, about 0.1% w/v to about 1% w/v of carbonic acid and water, wherein spray composition is stored in in high-pressure resistant Bag-On-Valve (BOV) aerosol dispensing system.

In another of the invention, the present invention provides the topical spray composition consisting about 0.01% w/v to about 1.0% w/v of chitosan, about 0.01% w/v to 2.0% w/v of lactic acid, about 0.1% w/v to about 1.0% w/v of carbonic acid and water quantity sufficient to 100% w/v of composition, wherein the spray composition has a pH of about 2.0 to about 4.5.

In another embodiment of the invention, the present invention provides the topical spray composition consisting about 0.01% w/v to about 1.0% w/v of chitosan, about 0.01% w/v to 0.2% w/v of lactic acid, about 0.1% w/v to about 1.0% w/v of carbonic acid and water, wherein the spray composition has a pH of about 2.0 to about 4.5 and wherein the spray composition is stored in a pressurized vessel.

In a further embodiment of the invention, the present invention provides the topical spray composition consisting about 0.01% w/v to about 1.0% w/v of chitosan, about 0.01% w/v to 0.2% w/v of lactic acid, about 0.1% w/v to about 1.0% w/v of carbonic acid and water, wherein the spray composition has a pH of about 2.0 to about 4.5 and wherein the spray composition is stored in high-pressure resistant container bottle with a nozzle in a canning mode.

In a still further embodiment of the invention, the present invention provides the topical spray composition consisting about 0.01% w/v to about 1.0% w/v of chitosan, about 0.01% w/v to 0.2% w/v of lactic acid, about 0.1% w/v to about 1% w/v of carbonic acid and water, wherein the spray composition has a pH of about 2.0 to about 4.5 and wherein spray composition is stored in in high-pressure resistant Bag-On-Valve (BOV) aerosol dispensing system.

The following example is provided to illustrate the present invention. It is understood, however, that the invention is not limited to the specific conditions or details described in the example below. The example should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

### Examples 1 to 6

Spray Composition:

| | Example 1 | Example 2 | Example 3 | Example 4 | Example 5 | Example 6 |
|---|---|---|---|---|---|---|
| Ingredient | (w/v) | (w/v) | (w/v) | (w/v) | (w/v) | (w/v) |
| Chitosan | 0.05% | 0.075% | 0.1% | 0.05% | 0.05% | 0.05% |
| Lactic acid | 0.04% | 0.07% | 0.14% | 0.04% | 0.04% | 0.04% |
| Carbonic acid | 0.4% | 0.4% | 0.4% | 0.4% | 0.4% | 0.4% |
| Glycerol | — | — | — | 1% | 3% | 5% |
| Water | Q.S to | Q.S to | Q.S to | Q.S to | Q.S to | Q.S to |
| | 100% | 100% | 100% | 100% | 100% | 100% |
| pH | 3.5 | 3.5 | 3.5 | 3.5 | 3.5 | 3.5 |

Process for Preparation of Examples 1-3
a. Collect 70% batch size of water for injection into suitable manufacturing vessel.
b. Add and disperse the chitosan in step a.
c. Add required quantity of 1 Molar lactic acid slowly till the chitosan get completely solubilized.
d. Makeup the volume to 100% batch size using water for injection.
e. Filter the solution through 0.2μ and cool to below 5° C.
f. Carbonate the solution and immediately fill into the container and closed.
Note: In case of non-aseptic filling, the product shall be sterilized by gamma irradiation or other suitable method of sterilization.

Process for Preparation of Examples 4-6
a. Collect 70% batch size of water for injection into suitable manufacturing vessel.
b. Add and disperse the chitosan in step-a.
c. Add required quantity of 1 Molar lactic acid slowly till the chitosan get completely solubilized.
d. Add dispensed quantity of glycerol to step c.
e. Makeup the volume to 100% batch size using water for injection.
f. Filter the solution through 0.2μ and cool to below 5° C.
g. Carbonate the solution and immediately fill into the container and closed.

Note: In case of non-aseptic filling, the product shall be sterilized by gamma irradiation or other suitable method of sterilization.

### Example-7: Procedure for Excision Wound Healing Study

Rats were anesthetized with ketamine (30 mg/kg, ip) and an area of about ≈500 mm² was marked on the back of the rat by a standard ring. Full thickness of the marked skin was then cut carefully. Treatment was started from 1^{st} day and continued till day 22. Wounds were traced on OHP sheet and calculated by superimposing on graph paper on the day of wounding and subsequently at a gap period of 4 days till 12^{th} day, then on the alternate days until healing was completed.

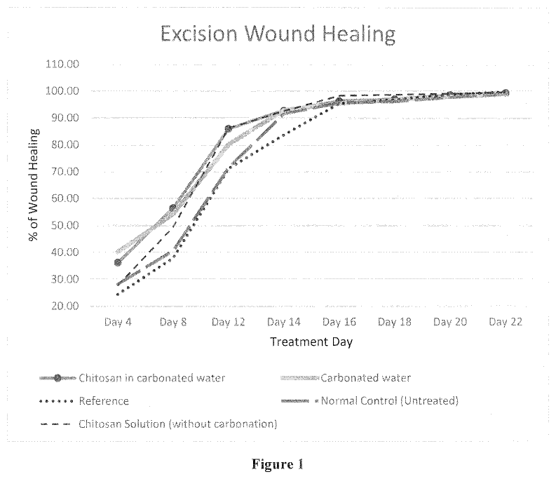

The changes in wound area was measured regularly and the rate of wound contraction calculated at the end of the study and the results are depicted in Table-1 and FIG. 1.

TABLE 1
Excision wound area - % Inhibition:

| | Treatment Day | | | | | | | |
|---|---|---|---|---|---|---|---|---|
| Formulation | 4 | 8 | 12 | 14 | 16 | 18 | 20 | 22 |
| Chitosan in carbonated water | 36.21 | 56.49 | 86.07 | 92.87 | 96.44 | 97.15 | 98.74 | 99.56 |
| (Example-1) | | | | | | | | |
| Chitosan Solution | 27.62 | 49.38 | 86.21 | 92.64 | 98.39 | 98.74 | 99.14 | 99.65 |
| (without carbonation) | | | | | | | | |
| Carbonated water | 40.30 | 54.20 | 80.20 | 92.99 | 95.48 | 96.83 | 97.97 | 99.02 |
| Reference Savlon ® | 24.37 | 37.83 | 71.18 | 83.69 | 95.47 | 96.90 | 98.90 | 99.73 |
| Normal Control (Untreated) | 28.23 | 40.59 | 71.52 | 91.59 | 95.83 | 96.23 | 97.79 | 99.17 |

### Example-8: Procedure for Incision Wound Healing Study

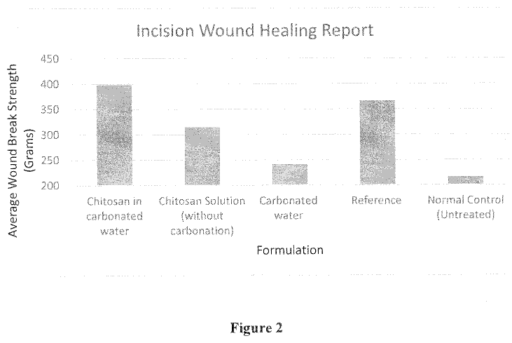

Two parallel six cm paravertebral incisions were made through the full thickness of the skin, 1 cm lateral to the midline of vertebral column after giving anesthesia. Wounds were closed with interrupted sutures, 1 cm apart, with surgical suture. The given formulations were applied topically for 10 days. The sutures were removed on the 7^{th} post-wounding day. Wound breaking strength was measured on the 10^{th} post-wounding day in anaesthetized rats. Wound breaking strength was determined by calculating the weight required to break the wound and the results are depicted in Table-2 and FIG. 2.

TABLE 2
Incision-wound healing:

| | Average Wound Break |
|---|---|
| Composition | Strength (Grams) |
| Chitosan in carbonated water (Example-1) | 398 |
| Chitosan Solution (without carbonation) | 314 |
| Carbonated water | 241 |
| Reference Savlon ® | 366 |
| Normal Control (Untreated) | 216 |

### Examples 9 to 12

Spray Composition:

| | Example 9 | Example 10 | Example 11 | Example 12 |
|---|---|---|---|---|
| Ingredient | (w/v) | (w/v) | (w/v) | (w/v) |
| Chitosan | 0.1% | 0.1% | 0.1% | 0.1% |
| Lactic acid | 0.5% | 0.5% | 1.2% | 1.2% |
| Carbonic acid | 0.2% | 0.3% | 0.2% | 0.3% |
| Water | Q.S to 100% | Q.S to 100% | Q.S to 100% | Q.S to 100% |
| pH | 2.96 | 2.96 | 2.58 | 2.61 |

Process for Preparation of Examples 9 to 12

Manufacturing Process A
a. Collect 110% purified water required for the batch in the manufacturing vessel.
b. Collect 90% of the purified water required for the batch in a separate vessel. Remaining quantity shall be used for volume make up.
c. To the step b, add Lactic acid under stirring.
d. To the step c, add Chitosan and stir until Chitosan is completely solubilized.
e. Volume made-up to 100% of the batch size with purified water.
f. Filter the solution through 0.2μ and cool to 2-10° C.
g. Carbonate the solution and immediately fill into the Bag-On-Valve aerosol dispensing system.

Manufacturing Process B
a. Collect 110% purified water required for the batch in the manufacturing vessel.
b. Collect 90% of the purified water required for the batch in a separate vessel. Remaining quantity shall be used for volume make up.
c. To the step b, add Chitosan under stirring to get homogenous dispersion.
d. To the step c, add Lactic acid and stir until Chitosan is completely solubilized.
e. Volume made-up to 100% of the batch size with purified water.
f. Filter the solution through 0.2μ and cool to 2-10° C.
g. Carbonate the solution and immediately fill into the Bag-On-Valve aerosol dispensing system.

### Example 13: Description, pH, Carbonic Acid (g/L) and Degree of De-Acetylation of Chitosan Testing of Example 12

Composition of Example-12 was stored at 40° C.±2° C./75±5% RH and tested for description, pH, carbonic acid (g/L) and degree of de-acetylation of chitosan for a period of six months and the results are depicted in Table-3.

TABLE 3

| | Tests | | | |
|---|---|---|---|---|
| | | | Carbonic | Degree of De- |
| | | | acid content | acetylation of |
| Time pointy | Description | pH | (g/L) | Chitosan (%) |
| Initial | Clear colorless | 2.61 | 3.08 | 86.2% |
| | solution | | | |
| 1 Month | Clear colorless | 2.57 | 3.02 | 85.5% |
| | solution | | | |
| 2 Months | Clear colorless | 2.52 | 2.97 | 86.3% |
| | solution | | | |
| 3 Months | Clear colorless | 2.58 | 2.99 | 87.1% |
| | solution | | | |
| 6 Months | Clear colorless | 2.52 | 3.06 | 86.4% |
| | solution | | | |

The above results indicate that the pH of the composition, carbonic acid content and degree of de-acetylation of chitosan is stable for a period of six months 40° C.±2° C./75±5% RH.

### Example 13: Carbonic Acid Content Evaluation of Example-12 Composition when Stored in High-Pressure Resistant Container Bottle with a Nozzle (Can) and Bag-On-Valve (BOV) Aerosol Dispensing System

Carbonic acid content (g/L) of Example-12 filled in High-pressure resistant container bottle with nozzle system (having propellant) and Bag-On-Value (BOV) Aerosol dispensing system was evaluated for a period of 10 days and results are depicted in Table-4.

TABLE 4

Carbonic acid content (g/L)

| | High-pressure resistant container | Bag- On-Valve |
|---|---|---|
| | bottle with a nozzle system | Aerosol (BOV) |
| | (containing composition of Example - | dispensing |
| Day | 12 mixed with propellant) | system |
| Day 1 | 3.02 | 3.03 |
| Day 2 | 2.99 | 3.01 |
| Day 3 | 3.01 | 3.02 |
| Day 4 | 2.96 | 2.99 |
| Day 5 | 2.94 | 2.96 |
| Day 6 | 2.90 | 2.97 |
| Day 7 | 2.60 | 2.95 |
| Day 8 | 2.45 | 2.94 |
| Day 9 | 2.31 | 2.96 |
| Day 10 | 2.15 | 2.91 |

The results indicate that the composition filled in BOV Aerosol dispensing system has better Carbonic acid content (g/L) throughout the usage period when compared to the composition filled in High-pressure resistant container bottle with a nozzle system (containing composition of Example-12 mixed with propellant).

1. A topical spray composition comprising chitosan and carbonic acid.

2. The topical spray composition according to claim 1, wherein the composition comprises about 0.01% w/v to about 1.0% w/v of chitosan.

3. The topical spray composition according to claim 1, wherein the composition comprises about 0.1% w/v to about 1.0% w/v of carbonic acid.

4. The topical spray composition according to claim 1, wherein the composition further comprises lactic acid and water.

5. The topical spray composition according to claim 1, wherein the spray composition is stored in a pressurized vessel.

6. The topical spray composition according to claim 5, wherein pressurized vessel is a Bag-On-Valve (BOV) aerosol dispensing system.

7. A topical spray composition comprising chitosan, carbonic acid, lactic acid and water.

8. The topical spray composition according to claim 7, wherein the composition comprises of about 0.01% w/v to about 1.0% w/v of chitosan, about 0.1% w/v to about 1.0% w/v of carbonic acid, 0.01% w/v to 2.0% w/v of lactic acid and water.

9. The topical spray composition according to claim 8, wherein the spray composition is stored in a pressurized vessel.

10. The topical spray composition according to claim 9, wherein pressurized vessel is a Bag-On-Valve (BOV) aerosol dispensing system.
